(19) United States
(12) Patent Application Publication
(10) Pub. No.: US 2025/0160628 A1
(43) Pub. Date: May 22, 2025

(54) IMAGING UNIT AND ENDOSCOPE

(71) Applicant: OLYMPUS MEDICAL SYSTEMS CORP., Hachioji-shi, Tokyo (JP)

(72) Inventors: Kazuyoshi Jingu, Inagi-shi (JP); Toshiyuki Fujii, Hachioji-shi (JP)

(73) Assignee: OLYMPUS MEDICAL SYSTEMS CORP., Hachioji-shi, Tokyo (JP)

(21) Appl. No.: 18/938,450

(22) Filed: Nov. 6, 2024

Related U.S. Application Data
(60) Provisional application No. 63/601,489, filed on Nov. 21, 2023.

(51) Int. Cl. A61B 1/00; A61B 1/05

(52) U.S. Cl. CPC A61B 1/00188; A61B 1/00096; A61B 1/0011; A61B 1/051

(57) ABSTRACT

An imaging unit includes a laminated lens obtained by laminating a plurality of lenses in an optical axis direction, and a metal film formed on a side circumferential surface of the laminated lens, in which the lenses constituting the laminated lens include at least one resin lens, the metal film is formed to cover at least a bonding portion side surface of the resin lens and another lens adjacent to the resin lens, the resin lens is bonded to the other adjacent lens with an adhesive, among the lenses constituting the laminated lens, the lens located on the side closest to an object is glass lens, and the lens on the image surface side from the glass lens is a resin lens.

### TECHNICAL FIELD

The present invention relates to an imaging unit and an endoscope. Priority is claimed on U.S. Provisional Application No. 63/601,489, filed Nov. 21, 2023, the content of which is incorporated herein by reference.

### BACKGROUND ART

An endoscope with an imaging unit having a laminated lens obtained by laminating multiple lens elements in an optical axis direction is known in the related art (e.g., refer to Patent Document 1). Discussion has been conducted with respect to such an endoscope to employ an imaging unit with a laminated lens composed of resin lenses laminated with no metal frame for low cost and a diameter reduction at the distal end of the insertion part of the endoscope for the next generation.

However, in the structure of such a resin laminated lens with no metal frame, moisture or a chemical solution permeating the resin lens or the resin tip frame (holding frame) penetrates into the laminated lens, which causes separation or clouding of the lenses of the laminated lens. For this reason, a structure that prevents moisture and chemical solutions from penetrating into laminated lenses has been demanded, and there is room for improvement in terms of this issue.

### CITATION LIST

### Patent Document

[Patent Document 1] Japanese Unexamined Patent Application, First Publication No. 2012-152390

### SUMMARY OF INVENTION

An imaging unit according to a first aspect of the present invention includes a laminated lens obtained by laminating a plurality of lenses in an optical axis direction, and a metal film formed on a side circumferential surface of the laminated lens, in which the lenses constituting the laminated lens include at least one resin lens, and the metal film is formed to cover at least a bonding portion side surface of the resin lens and another lens adjacent to the resin lens.

An endoscope according to a second aspect of the present invention has the above-described imaging unit mounted on a tip of a scope.

### BRIEF DESCRIPTION OF DRAWINGS

FIG. 1 is a perspective view of an endoscope according to a first embodiment.

FIG. 2 is a cross-sectional view of an imaging unit of the endoscope according to the first embodiment.

FIG. 3 is a cross-sectional view illustrating a method for creating a metal film according to the first embodiment.

FIG. 4 is a cross-sectional view illustrating the method for creating a metal film following FIG. 3 according to the first embodiment.

FIG. 5 is a cross-sectional view of an imaging unit of an endoscope according to a first variation.

FIG. 6 is a cross-sectional view of an imaging unit of an endoscope according to a second variation.

FIG. 7 is a cross-sectional view of an imaging unit of an endoscope according to a second embodiment.

FIG. 8 is a cross-sectional view of an imaging unit of an endoscope according to a third embodiment.

FIG. 9 is a cross-sectional view of an imaging unit of an endoscope according to a fourth embodiment.

FIG. 10 is a cross-sectional view of another imaging unit of the endoscope according to the fourth embodiment.

FIG. 11A is a cross-sectional view illustrating a method for creating a metal film according to the fourth embodiment.

FIG. 11B is a cross-sectional view illustrating a method for creating a metal film following FIG. 11A according to the fourth embodiment.

FIG. 12 is a cross-sectional view of an imaging unit of an endoscope according to a fifth embodiment.

FIG. 13 is a cross-sectional view illustrating a method for creating a metal film according to the fifth embodiment.

FIG. 14 is a cross-sectional view of an imaging unit of an endoscope according to a sixth embodiment.

FIG. 15 is a cross-sectional view illustrating a method for creating a metal film according to the sixth embodiment.

FIG. 16 is a cross-sectional view of an imaging unit of an endoscope according to a seventh embodiment.

FIG. 17 is a cross-sectional view of an imaging unit of an endoscope according to an eighth embodiment.

FIG. 18 is a cross-sectional view of an insertion part of an endoscope according to a ninth embodiment.

### DESCRIPTION OF EMBODIMENTS

### First Embodiment

An imaging unit and an endoscope according to embodiments of the present invention will be described below with reference to the drawings. Note that the scope of the present invention is not limited to the embodiments described below, and can be arbitrarily modified within the scope of the technical gist of the present invention. In addition, the actual structures, scales, numbers, and the like of each structure may vary in the following drawings in order to make each configuration easier to understand.

An endoscope 1 is a device that is inserted into a lumen of a patient to observe and treat lesions. In the following description, the side into which the endoscope 1 is inserted into a lumen of a patient will be referred to as a “distal end side (remote position side),” and the side opposite to the side into which the endoscope is inserted into a lumen of a patient will be referred to as a “proximal end side (close position side).”

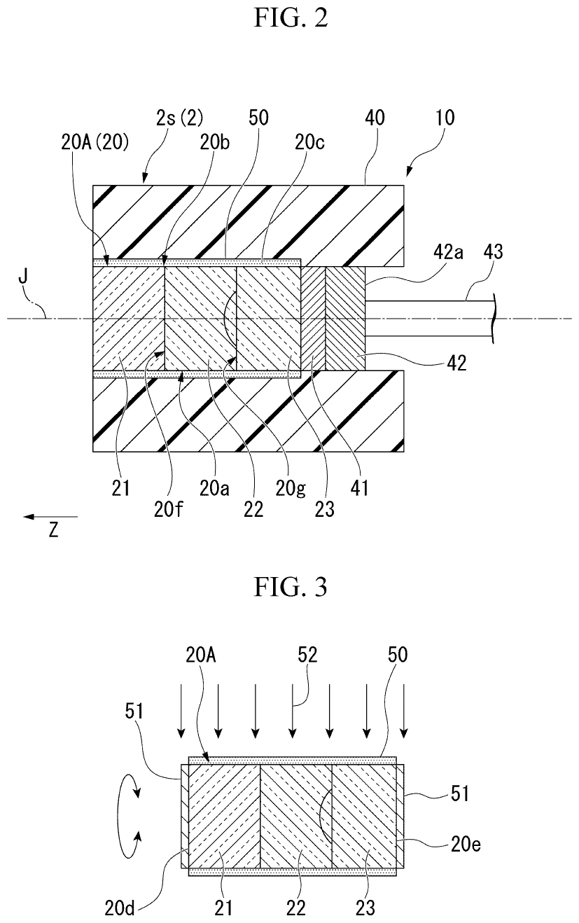

A Z axis is shown appropriately in each drawing. The Z axis represents the direction in which an optical axis J (refer to FIG. 2) of the embodiments to be described below extends. The optical axis J shown in each drawing is a virtual axial line. In the following description, the direction in which the optical axis J extends, that is, the direction parallel to the Z axis, will be referred to as an “optical axis direction.” A radial direction centered on the optical axis J will be referred to simply as a “radial direction.” A circumferential direction centered on the optical axis J will be referred to simply as a “circumferential direction.” The side in the optical axis direction to which an arrow of the Z axis is directed (+Z side) is the “distal end side (remote position side),” and the side in the optical axis direction opposite to the side to which the arrow of the Z axis is directed (−Z side) is the “proximal end side (close position side).” In the following description, the side in the optical axis direction to which the arrow of the Z axis is directed (+Z side) will be referred to as a “distal end side” or “the other side of the optical axis direction.” The side opposite to the side in the optical axis direction to which the arrow of the Z axis is directed (−Z side) will be referred to as a “proximal end side” or “the one side in the optical axis direction.”

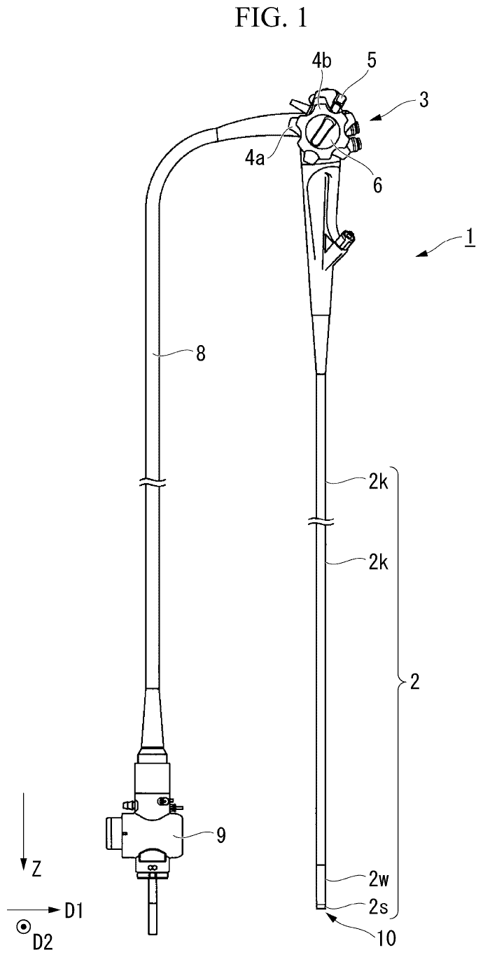

FIG. 1 is an exterior view illustrating the endoscope 1 with an imaging unit 10 according to the first embodiment. FIG. 2 is a cross-sectional view of a distal end part constituting an insertion part 2 of the endoscope 1.

As illustrated in FIG. 1, the endoscope 1 includes the insertion part 2 that is inserted into a medical subject, a manipulation part 3 that is connected to the proximal end side of the insertion part 2, a universal cord 8 that extends from the manipulation part 3, a connector 9 connected to the universal cord 8, and the imaging unit 10. The endoscope 1 is electrically connected to an external device such as a control device, a lighting device, and the like, which are not illustrated, through the connector 9.

The insertion part 2 has a distal end part 2s, a curve part 2w, and a flexible tube part 2k in this order from the distal end side. The insertion part 2 is formed in an elongated shape in the optical axis direction. The imaging unit 10 is provided at the tip of the distal end part 2s. That is, the imaging unit 10 is provided at the tip of the insertion part 2. The curve part 2w can be curved in each direction of a first direction D1 and a second direction D2 which are orthogonal to the optical axis direction. The flexible tube part 2k connects the curve part 2w and the manipulation part 3. The flexible tube part 2k has a cylindrical shape extending in the optical axis direction. An electrical cable 43 illustrated in FIG. 2 electrically connecting the connector 9 to the imaging unit 10 is housed in the flexible tube part 2k.

As illustrated in FIG. 1, the manipulation part 3 includes curve manipulation knobs 4a and 4b that cause the curve part 2w of the insertion part 2 to curve, a fixing lever 5 that fixes a rotating position of the curve manipulation knob 4a, a fixing knob 6 that fixes a rotating position of the curve manipulation knob 4b, a button for moving a moving frame (not illustrated) in the optical axis direction, and the like. By manipulating rotation of the curve manipulation knobs 4a and 4b, the curve part 2w curves in the first direction D1 and the second direction D2. Thus, the direction in which the imaging unit 10 observes can be changed, and the performance of insertion of the distal end part 2s into a medical subject can be improved. As will be described below, due to manipulation of the button, and the like (not illustrated), the moving frame can move in the optical axis direction, and thus focus on the subject can be switched.

As illustrated in FIG. 2, an objective lens (not illustrated) is disposed on the distal end surface of the insertion part 2. The objective lens is a lens of the imaging unit 10 functioning as a lens unit. The objective lens has the distal end surface exposed to the outer side of the endoscope 1, and is fixedly held by an objective lens holding part. A tip frame member of the insertion part 2 is made of, for example, stainless steel.

The imaging unit 10 has an optical system 20 with a moving lens (not illustrated) that is movable in the direction of the optical axis J, a moving frame (not illustrated) holding the moving lens, a driving part consisting of an electromagnetic actuator that drives the movement frame, a holding frame 40 including the moving frame, and a circuit board 42 for the driving part provided outside the holding frame 40. The driving part has a coil.

More detailed description is as follows. The moving lens that can advance and retreat in the optical axis direction (Z-axis direction) is disposed in the imaging unit 10 for focusing and zooming. The moving lens is fixedly held in the moving frame. The moving frame is driven to advance and retreat by the driving part that is a lens driving means.

The optical system 20 forms optical images of subjects. FIG. 2 schematically illustrates the imaging unit 10. In FIG. 2, the optical system 20 is constituted with a laminated lens 20A obtained by laminating three lenses 21, 22, and 23 in the optical axis direction. The laminated lens 20A has the first lens 21, the second lens 22, and the third lens 23 disposed in this order from the distal end side to the proximal end side. A configuration of the laminated lens 20A is not limited to the configuration of the present embodiment, and the number of lenses constituting the laminated lens 20A is, for example, four or more. In addition, a shape of the cross section of the laminated lens 20A is not particularly limited, and may be a rectangular shape or a circular shape.

The three lenses 21, 22, and 23 constituting the laminated lens 20A include at least one resin lens. In the present embodiment, the two lenses which are the second lens 22 and the third lens 23 are resin lenses. Among the three lenses 21, 22, and 23 constituting the laminated lens 20A, the first lens 21 located closest to the object is a glass lens. In other words, the lenses on the imaging surface side from the first lens 21 as a glass lens are resin lenses. The resin lenses of the second lens 22 and the third lens 23 are bonded to the first lens 21 as a glass lens adjacent thereto with an adhesive.

The first lens 21, the second lens 22, and the third lens 23 are held on the inner circumferential surface of the holding frame 40. An optical image of a subject formed by the optical system 20 is converted into an image signal by an image sensor 41, and transmitted to an external device via the circuit board 42 and the electrical cable 43.

A metal film 50 is formed on a side circumferential surface 20a of the laminated lens 20A. The metal film 50 is formed to cover at least a side surface of the bonding portion of the second lens 22 as a resin lens and another lens (the first lens 21) adjacent to the second lens 22 (a first bonding portion side surface 20b). Here, the first bonding portion side surface 20b is an area stretching to the first lens 21 and the second lens 22 and an area including a boundary surface 20f of the first lens 21 and the second lens 22. In the present embodiment, the metal film 50 covers the entire side circumferential surface 20a of the laminated lens 20A. For this reason, the metal film 50 also covers the side surface of the bonding portion of the second lens 22 and the third lens 23 as resin lenses (a second bonding portion side surface 20c). The second bonding portion side surface 20c is an area stretching to the second lens 22 and the third lens 23 and an area including a boundary surface 20g of the second lens 22 and the third lens 23.

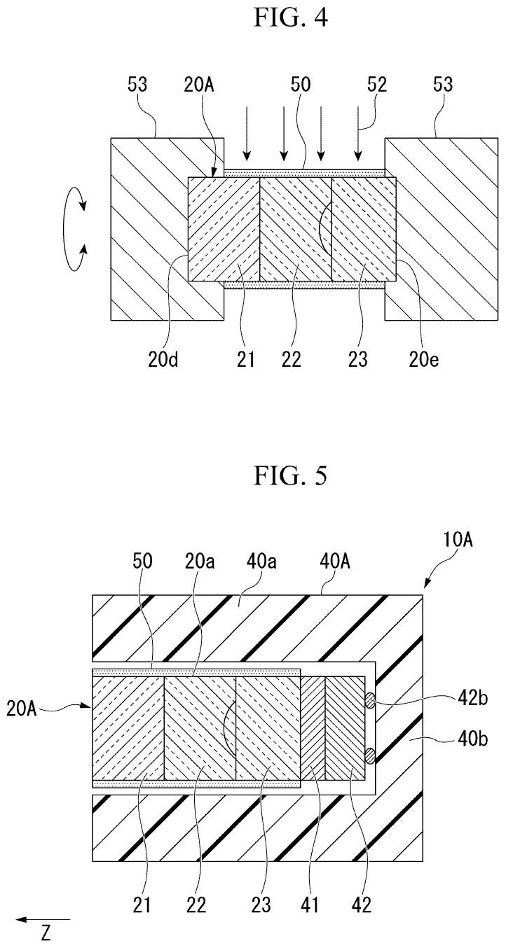

As a method for creating the metal film 50, there is a method of performing sputter deposition as illustrated in FIGS. 3 and 4. The example illustrated in FIG. 3 is a creation method for generating the metal film 50 in the entire circumference by covering both end surfaces 20d and 20e of the laminated lens 20A with a mask 51 and depositing a sputtering material 52 while rotating the laminated lens 20A. As the sputtering material 52, for example, gold, copper, aluminum, titanium, nickel, or the like can be employed.

The example illustrated in FIG. 4 is a creation method for generating the metal film 50 in the entire circumference by grasping the end surfaces 20d and 20e of the laminated lens 20A with a mask-cum-handle 53 and depositing the sputtering material 52 while rotating the laminated lens 20A.

Note that, a creation method of the metal film 50 is not limited to sputter deposition, and for example, a creation method performed by applying a silver paint, a conductive adhesive, or the like, or a creation method performed by wrapping a thin film such as aluminum foil, or a conductive heat-shrink tube can be employed.

The holding frame 40 (resin frame) has a tubular shape extending in the optical axis direction and covers the outer circumference of the laminated lens 20A in which the metal film 50 is formed as illustrated in FIG. 2. In the present embodiment, the holding frame 40 has a substantially cylindrical shape extending in the optical axis direction centered on the optical axis J. The holding frame 40 may have a prismatic shape extending in the optical axis direction. The holding frame 40 is open on both sides in the optical axis direction. The holding frame 40 houses a part of the optical system 20 inside. The holding frame 40 is formed of a resin member that is permeable to moisture, chemicals, and the like.

Each member, which is not illustrated, is housed in the distal end part 2s of the insertion part 2 in which the holding frame 40 is disposed. For example, the imaging unit 10, a light guide that supports a lighting lens, a channel tube for inserting a treatment instrument, a water injection nozzle, and a driving part for moving a moving frame are provided in the distal end part 2s of the insertion part 2. A treatment instrument that is inserted into the insertion part 2 and extends from the channel tube is a magnetic material. Note that the channel tube itself may be a magnetic material or a non-magnetic material.

The driving part is held on the outer side of the holding frame 40. The coil of the driving part is electrically connected to the circuit board 42. A current is supplied from an external device, which is not illustrated, to the driving part via the circuit board 42 and the electrical cable 43. In the present embodiment, the driving part is an electromagnetic actuator that moves the moving lens in the optical axis direction with a magnetic force. In the present embodiment, focus of the endoscope 1 on a subject can be switched by moving the moving lens in the optical axis direction. Note that a configuration of the driving part is not limited to the present embodiment, and the driving part may be, for example, a heater or the like that prevents condensation of the optical system 20.

In the present embodiment, a current supplied from an external device, which is not illustrated, to the driving part via the circuit board 42 and the electrical cable 43 is controlled in accordance with manipulation of a button.

The electrical cable 43 is electrically connected to an external device via the connector 9 illustrated in FIG. 1. The electrical cable 43 is electrically connected to the circuit board 42 as illustrated in FIG. 2. The coil of the driving part is electrically connected to an external device via the electrical cable 43 and the circuit board 42.

The image sensor 41 is fixed onto the inner circumferential surface of the holding frame 40 on the proximal end side. The image sensor 41 is an image sensor such as a CCD or a CMOS. The second lens 22 and the third lens 23 of the laminated lens 20A are adhesively fixed to the light-receiving surface side of the image sensor 41. The image sensor 41 converts an optical image of a subject formed by the optical system 20 into an image signal. The image sensor 41 is connected to an external device, which is not illustrated, via the electrical cable 43 and the connector 9 illustrated in FIG. 1. The electrical cable 43 electrically connected to the image sensor 41 extends from the holding frame 40 to the proximal end side. Multiple signal lines are inserted into the electrical cable 43.

The circuit board 42 electrically connects the coil to the electrical cable 43. Thus, the external device, which is not illustrated and the driving part are electrically connected, and a current is supplied from the external device to the coil. The circuit board 42 is disposed on the proximal end side of the image sensor 41. For example, the surface 42a of the circuit board 42 on the proximal end side is electrically connected to the electrical cable 43 by soldering.

The imaging unit 10 of the present embodiment has the laminated lens 20A obtained by laminating the multiple lenses 21, 22, and 23 in the optical axis direction, and the metal film 50 formed on the side circumferential surface 20a of the laminated lens 20A as described above. The lenses constituting the laminated lens 20A include at least one resin lens (here, the second lens 22 and the third lens 23). The metal film 50 is formed to cover at least the first bonding portion side surface 20b of the resin lens (the second lens 22 and the third lens 23) and the other lens (the first lens 21) adjacent thereto.

As a result, at least the first bonding portion side surface 20b of the laminated lens 20A is covered with the metal film 50 in the present embodiment, and thus penetration of moisture or a chemical solution into the side circumferential surface 20a of the laminated lens 20A can be prevented. Thus, the occurrence of separation and clouding of the lenses of the laminated lens 20A can be curbed.

In addition, since the present embodiment is of the configuration provided with the resin lenses, miniaturization and low cost of the imaging unit 10 can be achieved.

Furthermore, since the resin lenses (the second lens 22 and the third lens 23) are bonded to the other lens (the first lens 21) adjacent thereto with an adhesive in the present embodiment, the effect of static electricity on the outside of the endoscope 1 can be alleviated.

Moreover, among the lenses constituting the laminated lens 20A of the imaging unit 10 according to the present embodiment, the first lens 21 located closest to an object is a glass lens. For this reason, penetration of moisture and a chemical solution not only from the side circumferential surface 20a of the laminated lens 20A but also from the distal end of the laminated lens 20A can be prevented more reliably.

In addition, the imaging unit 10 according to the present embodiment has the holding frame 40 (resin frame) covering the outer circumference of the laminated lens 20A around which the metal film 50 is formed. Since the resin holding frame 40 covers the laminated lens 20A in this case, the effect of static electricity can be curbed in comparison to the case of a metal frame.

According to the imaging unit 10 and the endoscope 1 of the present embodiment, penetration of moisture and a chemical solution into the laminated lens 20A can be prevented, and the occurrence of separation and clouding of the lenses of the laminated lens 20A can be curbed.

Although the embodiment of the present invention has been described in detail above referring to the drawings, a specific configuration is not limited to the embodiment, and an amendment to a design that falls within the scope that does not depart from the gist of the present invention is also included. In addition, constituent elements introduced in the above-described embodiment and variations can be appropriately combined.

### First Variation

An imaging unit 10A according to a first variation has a configuration in which a circuit board 42 is directly connected to a resin holding frame 40A formed by injection molding as illustrated in FIG. 5. The holding frame 40A has a bottom wall 40b at the end of a tubular frame body 40a on the proximal end side in the optical axis direction. The circuit board 42 is connected to the bottom wall 40b by solder 42b. A laminated lens 20A is covered with a metal film 50 throughout a side circumferential surface 20a of the laminated lens 20A similarly to the above-described embodiment.

### Second Variation

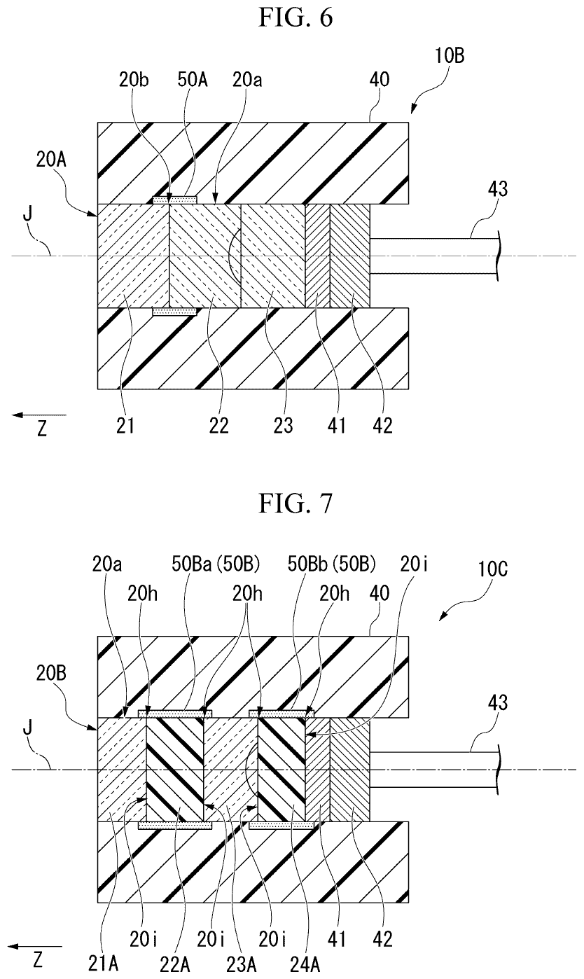

An imaging unit 10B according to a second variation is configured such that a metal film 50A covers only a first bonding portion side surface 20b of a second lens 22 as a resin lens and another lens (a first lens 21) adjacent to the second lens 22 as illustrated in FIG. 6.

In the imaging unit 10B according to the second variation, the first bonding portion side surface 20b that is close to the distal end part of a holding frame 40 and is likely to have a larger amount of moisture penetration can be reliably covered. In this case, since the metal film 50A is not provided on the entire side circumferential surface 20a of the laminated lens 20A, the material cost incurred for the metal film 50A can be reduced, and the spot expected to have a large amount of moisture penetration can be reliably covered with the metal film 50A.

### Second Embodiment

An imaging unit 10C according to a second embodiment includes a laminated lens 20B as illustrated in FIG. 7, which is different from the laminated lens 20A (see FIG. 2) according to the above-described first embodiment. The laminated lens 20B has a first lens 21A, a second lens 22A, a third lens 23A, and a fourth lens 24A disposed in this order from the distal end side to the proximal end side.

Among the four lenses 21A, 22A, 23A, and 24A of the laminated lens 20B, the two lenses which are the second lens 22A and the fourth lens 24A are resin lenses, and the two lenses which are the first lens 21A and the third lens 23A are glass lenses. The resin lenses as the second lens 22A and the fourth lens 24A are bonded to other lenses adjacent thereto with an adhesive.

Metal films 50B according to the second embodiment are provided at two spots in the optical axis direction. The metal film 50Ba on the distal end side is formed to cover a side surface of the second lens 22A as a resin lens and the side surfaces at the bonding portions of the resin lens and the glass lenses (third bonding portion side surfaces 20h) of the second lens 22A. The metal film 50Bb disposed on the proximal end side with an interval from the metal film 50Ba is formed to cover side surfaces of the fourth lens 24A as a resin lens and the third bonding portion side surfaces 20h of the fourth lens 24A. The third bonding portion side surface 20h is an area stretching to the resin lenses and glass lenses, and is an area including boundary surfaces 20i of the resin lenses and the glass lenses. The metal film 50B covers the entire side surfaces of the first lens 21A and the third lens 23A as glass lenses.

In the imaging unit 10C according to the second embodiment, the boundary surfaces 20i between the resin lenses and the glass lenses that are different material can be reliably covered. In addition, since the metal film 50B is not provided through the side circumferential surface 20a of the laminated lens 20B, the cost for the material needed in the metal frame 50B can be reduced.

### Third Embodiment

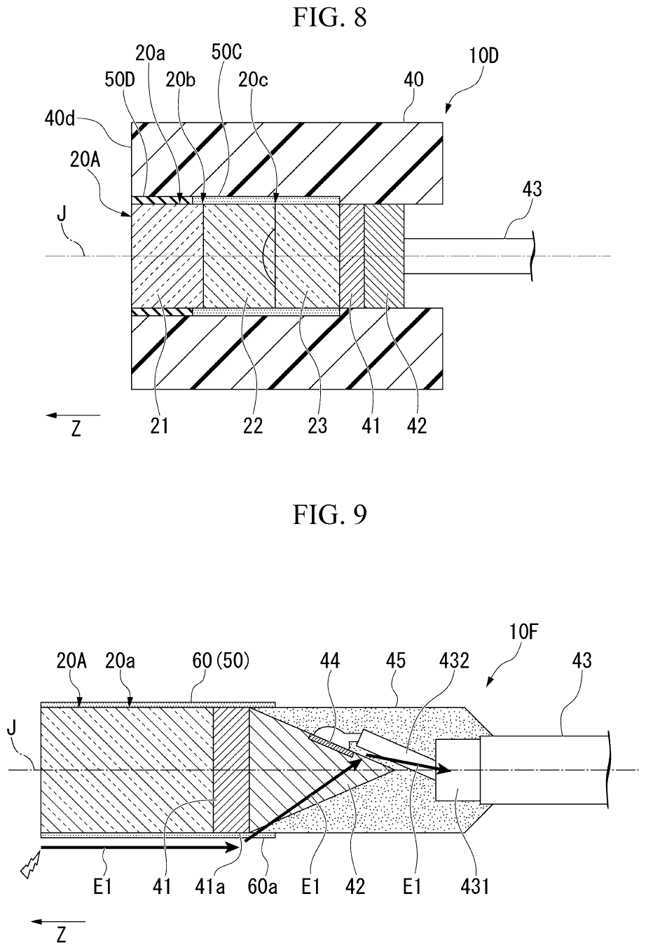

As illustrated in FIG. 8, an imaging unit 10D according to a third embodiment is configured such that in the laminated lens 20A similar to that of the above-described first embodiment, the side circumferential surface 20a of the laminated lens 20A is covered with a metal film 50C and an insulating resin film 50D. In the imaging unit 10D, a side circumferential surface of the first lens 21 as a glass lens forms an insulating resin film 50D, a second lens 22 and a third lens 23 as resin lenses on the proximal end side from the insulating resin film 50D form the metal film 50C, and the side surface at the bonding portion of the first lens 21 and the second lens 22 (a first bonding portion side surface 20b) is covered with the metal film 50C. The first bonding portion side surface 20b and a second bonding portion side surface 20c are covered with the metal film 50C. The portion of the first lens 21 on the distal end side on which the metal film 50C is not formed adheres to a holding frame 40 via the insulating resin film 50D made of an insulating resin.

Since the imaging unit 10D according to the third embodiment has a configuration in which the portion of the first lens 21 at the distal end side adheres to the holding frame 40 via the insulating resin film 50D made of an insulating resin and metal of the metal film 50C is not exposed to a distal end surface 40d of the holding frame 40, the effects of static electricity can be curbed.

### Fourth Embodiment

As illustrated in FIG. 9, an imaging unit 10F according to a fourth embodiment includes a conductive film 60 made of a conductive member electrically connecting a GND terminal of an image sensor 41 provided on the imaging surface side of the laminated lens 20A and a metal film 50. The imaging unit 10F has an electrical cable 43 electrically connected by soldering, etc. via a cable connection terminal 44 at a predetermined position of a circuit board 42 provided at the proximal end side of the image sensor 41. In addition, the circuit board 42, and a shield 431 and a signal line 432 of the electrical cable 43 are covered with a reinforcing adhesive part 45 formed by curing an adhesive.

Although the metal film 50 and the conductive film 60 are formed of the same member in the present embodiment, the metal film 50 and the conductive film 60 may be provided separately. The conductive film 60 has a thickness of, for example, 10 μm or less. The conductive film 60 is not limited to covering an entire side circumferential surface 20a of the laminated lens 20A, similarly to the metal film 50 according to the above-described embodiment. A proximal end part 60a of the conductive film 60 covers at least a portion of an outer circumferential surface 41a of the image sensor 41. Although the entire outer circumferential surface 41a of the image sensor 41 is covered with the conductive film 60 without gaps in the present embodiment, the outer circumferential surface 41a may be partially covered with the conductive film 60.

The conductive film 60 serves as a path for static electricity by connecting to GND wiring on the circuit board 42. The arrow E1 illustrated in FIG. 9 indicates a static electricity conduction path, indicating a state in which static electricity generated at the distal end side of the laminated lens 20A escapes to the ground (GND) via the conductive film 60. In other words, the conductive film 60, the circuit board 42, the cable connection terminal 44, the shield 431 of the electrical cable 43, and GND serve as the static electricity conduction path E1.

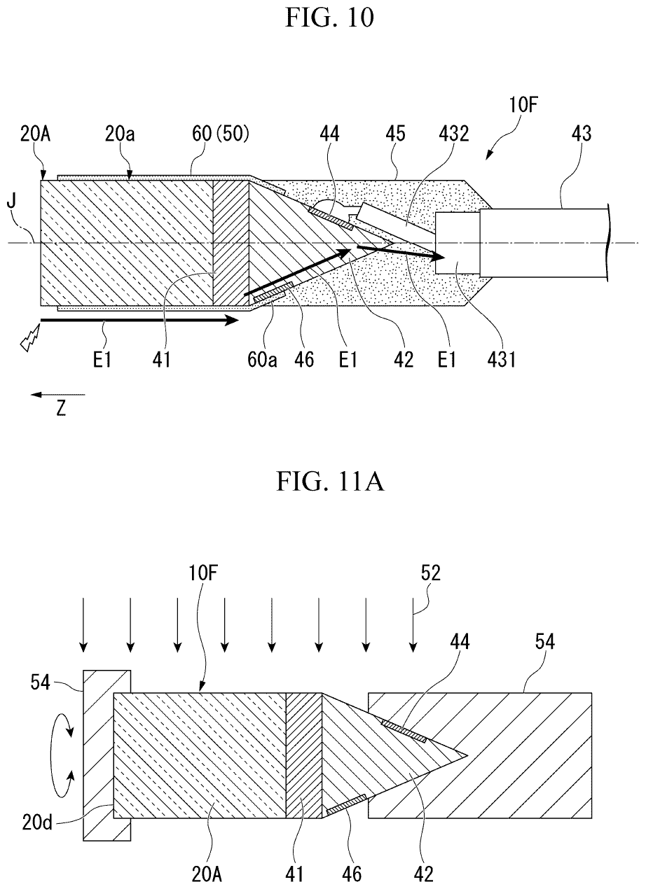

FIG. 10 illustrates a configuration of an imaging unit 10F in which a ground terminal (GND terminal 46) is provided on the circuit board 42 and the GND terminal 46 is made conductive as it is covered with a proximal end part 60a of the conductive film 60. The GND terminal 46 is disposed on a distal end side from the cable connection terminal 44. In this case, the conductive film 60, the GND terminal 46, the circuit board 42, the cable connection terminal 44, the shield 431 of the electrical cable 43, and the GND serve as the static electricity conduction path E1.

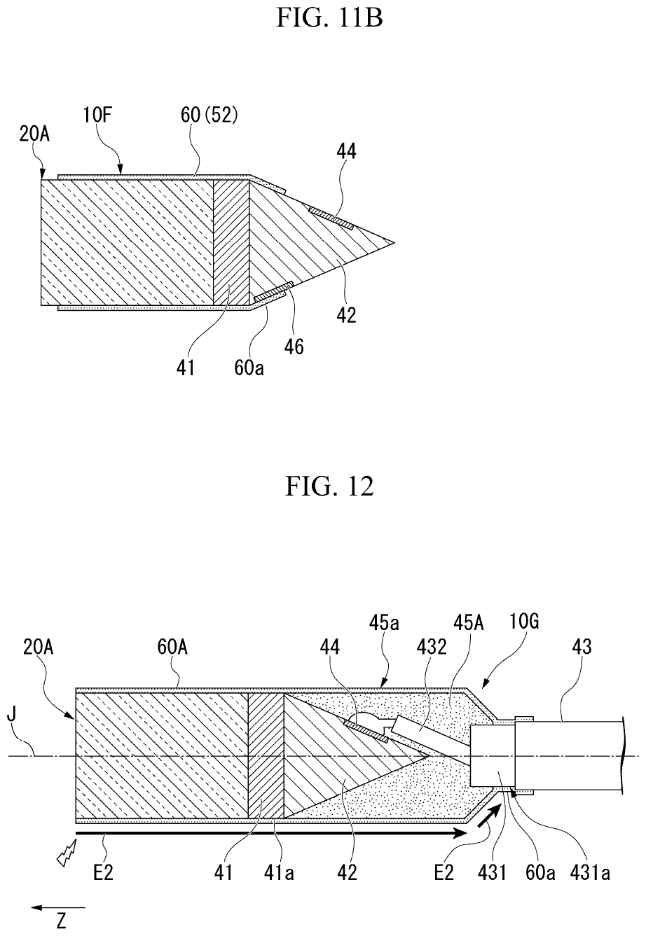

As a method for creating the conductive film 60 of the imaging unit 10F, there is a method of performing sputter deposition as illustrated in FIGS. 11A and 11B. As illustrated in FIG. 11A, after the laminated lens 20A, the image sensor 41, and the circuit board 42 are integrally formed, each of the distal end surface 20d of the laminated lens 20A and a portion of the circuit board 42 on the proximal end side is gripped with a gripping/masking jig 54. At this time, the cable connection terminal 44 is in a state of being masked by the gripping/masking jig 54, and the GND terminal 46 is in an exposed state. After that, by depositing sputtering material 52 while rotating the laminated lens 20A, the conductive film 60 is generated around the entire circumference (see FIG. 11B). As the sputtering material 52, for example, gold, copper, aluminum, titanium, nickel, or the like can be employed. Thus, the sputtering material 52 is coated on the GND terminal 46 as illustrated in FIG. 11B, and thus the proximal end part 60a of the sputtering material 52 (conductive film 60) and the GND terminal 46 become conductive.

Note that, a creation method of the conductive film 60 is not limited to sputter deposition, and a creation method performed by applying a silver paint, a conductive adhesive, or the like, or a creation method performed by wrapping a thin film such as aluminum foil, or a conductive heat-shrink tube can be employed.

In the imaging unit 10F according to the fourth embodiment, since the conductive film 60 formed by the sputtering material 52 is electrically connected to the image sensor 41, the conductive film 60 serves as a path for static electricity generated at the distal end side of the laminated lens 20A, and thus the static electricity can be allowed to escape to the GND via the circuit board 42 and the electrical cable 43. In the present embodiment as described above, an escape route of static electricity can be efficiently formed with a simple structure in the imaging unit 10F in which the laminated lens 20A requiring measures against static electricity is mounted.

Furthermore, since measures against static electricity can be taken in the imaging unit 10F by forming the thin conductive film 60 as the static electricity conduction path E1 without gaps for the laminated lens 20A and the image sensor 41 without providing a metallic frame member (metal frame), the imaging unit 10F and the endoscope 1 can be miniaturized. In other words, in the imaging unit 10F according to the present embodiment, no gap for fitting a metal frame is required in the space from the laminated lens, unlike in the related art, and moreover, a thick large metal frame is not needed. Furthermore, when a metal frame is provided, jumper wires need to have a size in consideration of manufacturability, which makes jumper wire connection parts prone to an increase in size, and thus the effect of miniaturization of the imaging unit 10F brought by eliminating the need for a metal frame as in the present embodiment is enormous.

### Fifth Embodiment

An imaging unit 10G according to a fifth embodiment is configured such that, in the imaging unit 10F of the above-described fourth embodiment (see FIG. 10), a proximal end part 60a of a conductive film 60A covers a part of a shield 431 (an exposed part 431a to be described below) of an electrical cable 43 to be electrically connected thereto as illustrated in FIG. 12. In this case, because the conductive film 60A is coated on an outer circumferential surface 41a of an image sensor 41, it is in conduction with the image sensor 41. The shield 431 has a portion embedded in a reinforcing adhesive part 45A on the distal end side and a portion on a proximal end side serving as the exposed part 431a not covered with the reinforcing adhesive part 45A. This exposed part 431a is covered with the proximal end part 60a of the conductive film 60A. In addition, the reinforcing adhesive part 45A has an outer circumferential surface 45a entirely covered with the conductive film 60A coated by sputtering.

The conductive film 60A serves as a path for static electricity by connecting to GND wiring of the exposed part 431a of the shield 431. A static electricity conduction path E2 is a path formed of the conductive film 60A and the shield 431 (GND wiring) of the electrical cable 43, through which static electricity generated at the distal end side of the laminated lens 20A escapes to the ground (GND) via the conductive film 60A.

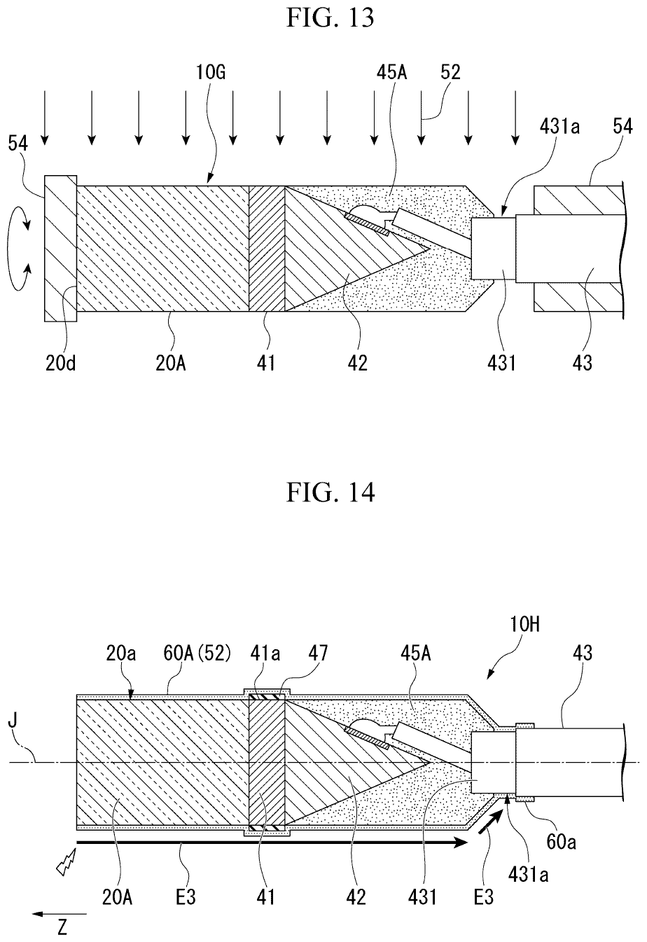

As a method for creating the conductive film 60A of the imaging unit 10G, there is a method of performing sputter deposition as illustrated in FIG. 13. After the laminated lens 20A, the image sensor 41, the circuit board 42, the electrical cable 43, and the reinforcing adhesive part 45A are integrally formed, each of the distal end surface 20d of the laminated lens 20A and a portion of the electrical cable 43 is gripped with a gripping/masking jig 54. At this time, the exposed part 431a of the shield 431 of the electrical cable 43 becomes exposed. After that, by depositing a sputtering material 52 while rotating the laminated lens 20A, the conductive film 60A is generated around the entire circumference. Accordingly, the exposed part 431a of the shield 431 is coated with the sputtering material 52, and thus the sputtering material 52 (conductive film 60A) is in conduction with the shield 431 as illustrated in FIG. 12.

In the imaging unit 10G according to the fifth embodiment, since the sputtering material 52A is connected to the shield 431 of the electrical cable 43, the conductive film 60A serves as a path for static electricity generated at the distal end side of the laminated lens 20A, and thus the static electricity can be allowed to escape to the GND. In the present embodiment as described above, an escape route of static electricity can be efficiently formed with a simple structure in the imaging unit 10G in which the laminated lens 20A requiring measures against static electricity is mounted.

### Sixth Embodiment

As illustrated in FIG. 14, an imaging unit 10H according to a sixth embodiment is configured such that the outer circumferential surface 41a of the image sensor 41 is entirely covered with an insulating resin 47 in the imaging unit 10G according to the fifth embodiment (see FIG. 12). The outside of the insulating resin 47 is coated with the conductive film 60A. The conductive film 60A serves as a path for static electricity by connecting to GND wiring of the exposed part 431a of the shield 431 in the imaging unit 10H of the present embodiment. That is, a static electricity conduction path E3 is a path formed of the conductive film 60A and the shield 431 (GND wiring) of the electrical cable 43, through which static electricity generated at the distal end side of the laminated lens 20A escapes to the ground (GND) via the conductive film 60A.

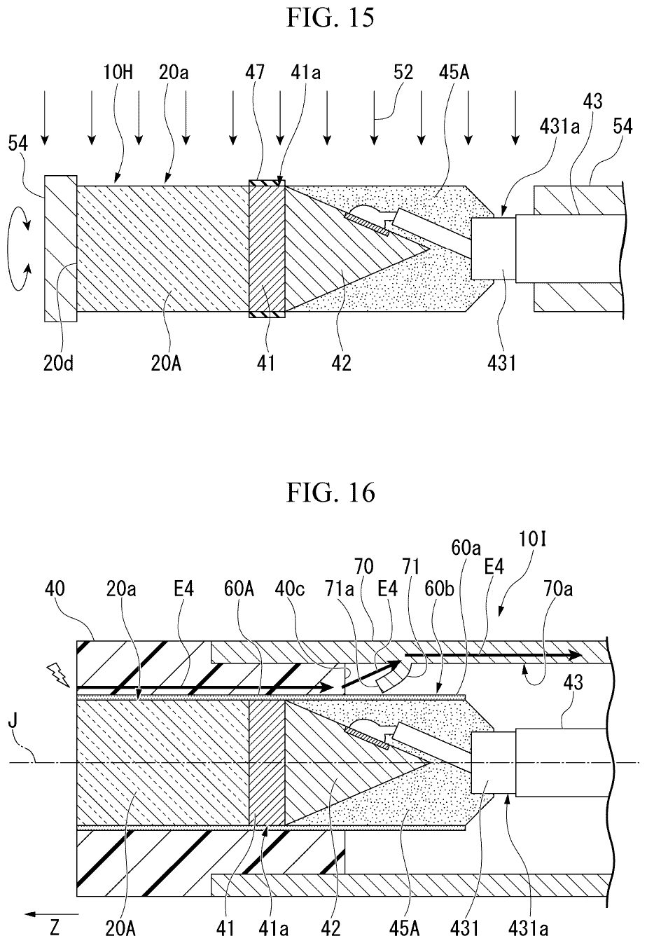

As a method for creating the conductive film 60A of the imaging unit 10H, there is a method of performing sputter deposition as illustrated in FIG. 15. After the laminated lens 20A, the image sensor 41, the circuit board 42, the electrical cable 43, and the reinforcing adhesive part 45A are integrally formed, each of the distal end surface 20d of the laminated lens 20A and a portion of the electrical cable 43 is gripped with a gripping/masking jig 54. At this time, the exposed part 431a of the shield 431 of the electrical cable 43 becomes exposed. First, the insulating resin 47 is applied to the entire outer circumferential surface 41a of the image sensor 41 while rotating the laminated lens 20A. After that, by depositing the sputtering material 52 while rotating the laminated lens 20A, the conductive film 60A (see FIG. 14) is generated around the entire circumference. Accordingly, the exposed part 431a of the shield 431 is coated with the sputtering material 52, and thus the sputtering material 52 (conductive film 60A) is in conduction with the shield 431 as illustrated in FIG. 14.

It should be noted that a procedure of applying the insulating resin 47 to the entire outer circumferential surface 41a of the image sensor 41 of the integrated imaging unit 10H, gripping the imaging unit with the gripping/masking jig 54, and depositing the sputtering material 52 may be employed.

Since the insulating resin 47 is applied to the outer circumferential surface 41a of the image sensor 41 and the image sensor 41 is not in conduction with the conductive film 60A in the imaging unit 10H of the sixth embodiment, static electricity can be prevented from flowing into the insulating resin 47 through the conductive film 60A.

In addition, since the electrical distance of the image sensor 41 to the escape route of static electricity increases in the present embodiment, resistance of the image sensor 41 against static electricity can be improved.

### Seventh Embodiment

An imaging unit 10I according to a seventh embodiment is configured such that, in the imaging unit 10F of the above-described fourth embodiment (see FIG. 9), the proximal end part 60a of the conductive film 60A covers a part of a reinforcing adhesive part 45A and is electrically connected to a metal frame 70 provided around the laminated lens 20A as illustrated in FIG. 16. The reinforcing adhesive part 45A embeds only the distal end side of the shield 431 of the electrical cable 43. For this reason, the portion of the shield 431 at the proximal end side constitutes an exposed part 431a not covered with the reinforcing adhesive part 45A. A side circumferential surface 20a of the laminated lens 20A to the reinforcing adhesive part 45A are coated with the conductive film 60A. Because the conductive film 60A is coated on the outer circumferential surface 41a of an image sensor 41, it is in conduction with the image sensor 41.

The metal frame 70 is a metallic tube having a C-shaped cross-section. The metal frame 70 is fitted to the outer side of the holding frame 40 at the proximal end side. The conductive film 60A covering the outer circumferential surface of the reinforcing adhesive part 45A protrudes to the proximal end side beyond the proximal end part 40c of the holding frame 40. The protruding portion (a conductive exposed part 60b) of the conductive film 60A faces an inner surface 70a of the metal frame 70 having a gap therebetween. A claw part 71 protruding toward the conductive exposed part 60b is provided on the inner surface 70a of the metal frame 70. A tip 71a of the claw part 71 comes in contact with the conductive exposed part 60b. The claw part 71 may be provided at a part of the circumference of the metal frame 70, or may be provided along the entire length thereof in the circumferential direction.

The conductive film 60A serves as a path for static electricity by connecting to the metal frame 70 in the imaging unit 10I according to the seventh embodiment. A static electricity conduction path E4 is a path formed of the conductive film 60A, the claw part 71, and the metal frame 70, through which static electricity generated at the distal end side of the laminated lens 20A escapes to the ground (GND) from the conductive film 60A via the metal frame 70. In the present embodiment as described above, an escape route of static electricity can be efficiently formed with a simple structure in the imaging unit 10I in which the laminated lens 20A requiring measures against static electricity is mounted.

### Eighth Embodiment

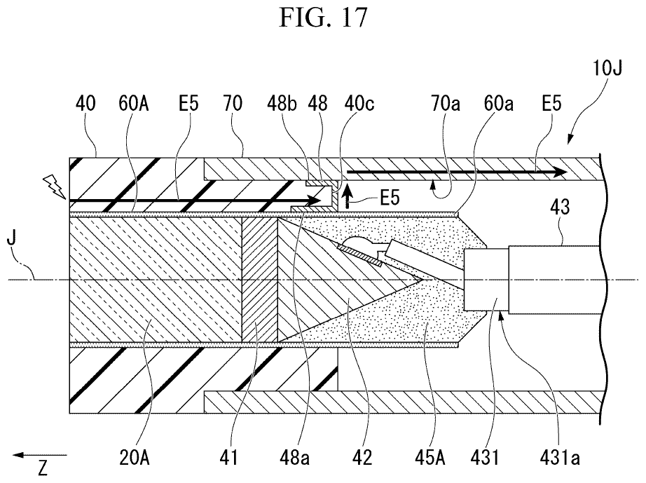

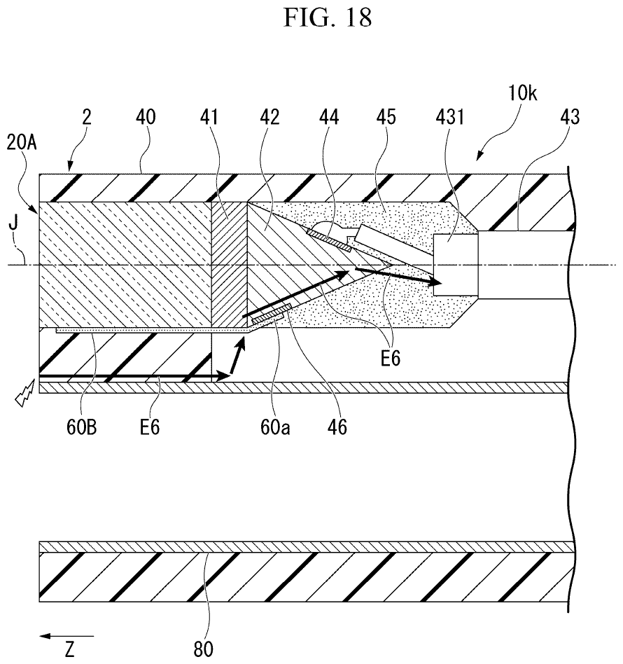

An imaging unit 10J according to an eighth embodiment is configured such that, in the imaging unit 10I of the above-described seventh embodiment (see FIG. 16), the proximal end part 60a of the conductive film 60A covers a part of the reinforcing adhesive part 45A and is electrically connected to the metal frame 70 provided around the laminated lens 20A as illustrated in FIG. 17. The conductive film 60A covering the outer circumferential surface of the reinforcing adhesive part 45A protrudes to the proximal end side beyond the proximal end part 40c of the holding frame 40. The protruding portion (a conductive exposed part 60b) of the conductive film 60A faces an inner surface 70a of the metal frame 70 having a gap therebetween.

A wiring part 48 electrically connecting the conductive film 60A and the metal frame 70 is formed at the proximal end part 40c of the holding frame 40. The wiring part 48 has an end part 48a coming in contact with the conductive film 60A and the other end part 48b coming in contact with the inner surface 70a of the metal frame 70.

The conductive film 60A serves as a path for static electricity by connecting to the metal frame 70 via the wiring part 48 in the imaging unit 10J according to the eighth embodiment. A static electricity conduction path E5 is a path formed of the conductive film 60A, the wiring part 48, and the metal frame 70, through which static electricity generated at the distal end side of the laminated lens 20A escapes to the ground (GND) from the conductive film 60A via the metal frame 70. In the present embodiment as described above, an escape route of static electricity can be efficiently formed with a simple structure in the imaging unit 10J in which the laminated lens 20A requiring measures against static electricity is mounted.

### Ninth Embodiment

As illustrated in FIG. 18, an imaging unit 10K according to a ninth embodiment is configured such that a conductive film 60B partially covers the imaging unit 10F according to the above-described fourth embodiment illustrated in FIG. 10 in the circumferential direction. A ground terminal (GND terminal 46) is provided on the circuit board 42 and the GND terminal 46 is made conductive as it is covered with the proximal end part 60a of the conductive film 60. The GND terminal 46 is disposed on a distal end side from the cable connection terminal 44.

A metallic cap member 80 having a C-shaped cross-section is provided near the conductive film 60B in parallel in the optical axis direction in an insertion part 2. The cap member 80 is a conductor to which static electricity is easily applied. In addition, the conductive film 60B is formed only in a close region facing the cap member 80 as a conductor.

In this case, the cap member 80, the conductive film 60B, the GND terminal 46, the circuit board 42, the cable connection terminal 44, the shield 431 of the electrical cable 43, and the GND serve as a static electricity conduction path E6. That is, the static electricity conduction path E6 is a path through which static electricity generated at the distal end side of the cap member 80 escapes to the ground (GND) from the conductive film 60B having static electricity applied thereto via the circuit board 42 and the electrical cable 43.

Since the ninth embodiment is a configuration in which the conductive film 60B is not disposed in the portion to which static electricity from the cap member 80 is difficult to be applied, the simple configuration is advantageous.

Although exemplary embodiments of the present invention have been described above, the present invention is not limited thereto. Additions, omissions, substitutions, and other modifications of the configuration are possible within the scope not departing from the gist of the present invention.

In addition, the present invention is not limited by the above description, and is limited only by the scope of the appended claims.

### INDUSTRIAL APPLICABILITY

The present invention can be applied to an imaging unit and an endoscope.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

1 Endoscope
2 Insertion part
2s Distal end part
10, 10A to 10K Imaging unit
20 Optical system
20A, 20B Laminated lens
20A Side circumferential surface
20b First bonding portion side surface
20c Second bonding portion side surface
20h Third bonding portion side surface
21 First lens (glass lens)
22 Second lens (resin lens)
23 Third lens (resin lens)
21A First lens (glass lens)
22A Second lens (resin lens)
23A Third lens (glass lens)
24A Fourth lens (resin lens)
40, 40A Holding frame (resin frame)
41 Image sensor
41a Outer circumferential surface
42 Circuit board
43 Electrical cable
431 Shield
44 Cable connection terminal
45, 45A Reinforcing adhesive part
46 GND terminal
47 Insulating resin
50, 50A, 50B, 50C Metal film
52 Sputtering material
60, 60A, 60B Conductive film
70 Metal frame
71 Claw part
E1 to E6 Static electricity conduction path
J Optical axis

What is claimed is:

1. An imaging unit comprising:
a laminated lens obtained by laminating a plurality of lenses in an optical axis direction; and
a metal film formed on a side circumferential surface of the laminated lens,
wherein the lenses constituting the laminated lens include at least one resin lens, and
the metal film is formed to cover at least a bonding portion side surface of the resin lens and another lens adjacent to the resin lens.

2. The imaging unit according to claim 1, wherein the resin lens is bonded to the other adjacent lens with an adhesive.

3. The imaging unit according to claim 1, wherein, among the lenses constituting the laminated lens, the lens located on a side closest to an object is a glass lens.

4. The imaging unit according to claim 3, wherein an insulating resin is formed on a side circumferential surface of the glass lens.

5. The imaging unit according to claim 1, comprising a resin frame covering an outer circumference of the laminated lens around which the metal film is formed.

6. The imaging unit according to claim 1, further comprising:
an image sensor provided on an imaging surface side of the laminated lens; and
a conductive member provided to connect the metal film and a GND terminal of the image sensor.

7. The imaging unit according to claim 1, comprising a conductive member provided to connect the metal film and a metal frame provided around the laminated lens.

8. The imaging unit according to claim 1, wherein the metal film is formed by sputtering.

9. An endoscope configured by mounting the imaging unit according to any one of claims 1 to 8 on a tip of a scope.
